(19) United States
(12) Patent Application Publication
(10) Pub. No.: US 2002/0182622 A1
(43) Pub. Date: Dec. 5, 2002

(54) Method for SNP (single nucleotide polymorphism) typing

(76) Inventors: Yusuke Nakamura, Kanagawa (JP); Toshihiro Tanaka, Tokyo (JP); Yozo Ohnishi, Tokyo (JP); Koichi Ozaki, Tokyo (JP); Ryo Yamada, Tokyo (JP)

Correspondence Address: BIRCH STEWART KOLASCH & BIRCH, PO BOX 747, FALLS CHURCH, VA, 22040-0747, US

(21) Appl. No.: 10/060,301

(22) Filed: Feb. 1, 2002

(30) Foreign Application Priority Data
Feb. 1, 2001 (JP) 25700/2001

(51) Int. Cl. C12Q 1/68; C12P 19/34

(52) U.S. Cl. 435/6; 435/91.2

(57) ABSTRACT

The present invention enables typing nearly hundreds of thousands of SNP sites using remarkably a small amount of genomic DNA. That is, the method for SNP typing of the present invention comprises the steps of simultaneously amplifying a plurality of nucleotide sequences comprising at least one or more sites of single nucleotide polymorphism (SNP) using genomic DNA and a plurality of primer pairs; and typing for distinguishing nucleotides of SNP sites contained in the plurality of nucleotide sequences amplified by the above amplification step, using the amplified nucleotide sequences.

### FIELD OF THE INVENTION

The present invention relates to a method for single nucleotide polymorphism (so-called “SNP”) typing which is used for identifying polymorphism of a site of SNP in genomic DNA.

### BACKGROUND OF THE INVENTION

Just as human appearances vary widely, their 3 billion genetic codes differ at numerous sites when compared between individuals. Such differences in genetic codes are called polymorphisms. A single nucleotide polymorphism (hereinafter referred to as “SNP”) is known as a typical polymorphism.

SNP, single nucleotide polymorphism, means a single base difference among a plurality of individuals. SNPs are classified into cSNP (coding SNP) and gSNP (genome SNP) according to their location. cSNP further includes sSNP (silent SNP), rSNP (regulatory SNP) and iSNP (intron SNP). In this specification, a site at which SNP occurs in genomic DNA, that is, a certain nucleotide at which SNP occurs is referred to as “single nucleotide polymorphism site” or “SNP site.”

Three to ten million SNP sites are thought to exist in the human genome. It is thought that some of these SNPs affect the control of expression or functions of proteins, and some involve individual differences in body compositions and susceptibility to a disease. That is, made to order medical care can be given according to an individual's body composition by obtaining information on SNPs. Accordingly, SNPs are increasingly discovered and identified, and many SNPs have already been reported.

The next task is to analyze information about how each SNP affects responsiveness to drugs and diseases, body composition or the like. To accomplish the task, SNP typing (a process to discriminate nucleotides of SNP sites) must be done to know how each individual's SNPs are. Such SNP typing would be a large-scale analytical process, such that SNP typing is performed for several hundreds of thousands of SNP sites per individual.

Examples of SNP typing are those using genomic DNA, including a TaqMan PCR method, an invader assay, a SniPer method, a MALDI-TOF-MASS method, a DNA chip method and the like. All of these methods require at least several tens of nanograms of genomic DNA to genotype one SNP site. When several hundreds of thousands of SNP sites are genotyped, several mg of genomic DNA is required per individual.

Nearly 11 of blood must be collected from an individual to obtain several mg of genomic DNA, however, it is actually impossible to obtain genomic DNA in such a volume from an individual. That is, typing of hundreds of thousands of SNP sites per individual cannot be performed at one time.

### SUMMARY OF THE INVENTION

We have completed the present invention under these circumstances. The purpose of the present invention is to provide a method for SNP typing which can genotype hundreds of thousands of SNP sites using a remarkably small amount of genomic DNA.

A method for SNP typing according to the present invention, which has achieved the above purposes, comprises the steps of simultaneously amplifying a plurality of nucleotide sequences comprising at least one or more SNP sites using genomic DNA and a plurality of primer pairs; and typing to discriminate nucleotides of SNP sites contained in the plurality of the nucleotide sequences amplified by the above amplification step.

In the method for SNP typing according to the present invention, a polymerase chain reaction using a hot start method is preferably used in the above amplification step.

Further in the method for SNP typing according to the present invention, 50 or more primer pairs are preferably used in the above amplification step.

Furthermore in the method for SNP typing according to the present invention, an Invader assay or a TaqMan PCR method is preferably used in the above typing step.

Furthermore in the method for SNP typing according to the present invention, an amplification is carried out from 10 ng to 40 ng of genomic DNA.

### DETAILED DESCRIPTION OF THE INVENTION

Now the method for SNP typing according to the present invention will be further described in detail.

In the method for SNP typing according to the present invention, first, an amplification step is performed to simultaneously amplify a plurality of nucleotide sequences comprising at least one or more SNP sites using genomic DNA to be analyzed and a plurality of primer pairs. Then a typing step is performed for typing using the nucleotide sequences amplified in the amplification step.

1. Amplification Step:

In the method of the present invention, genomic DNA to be analyzed can be extracted by using standard, known techniques. Genomic DNA to be analyzed can also be extracted by isolating leucocytes from peripheral blood collected from a human, and extracting according to standard techniques from the isolated leucocytes. Particularly when hundreds of thousands of SNP sites are genotyped, approximately several tens of micrograms of genomic DNA is prepared in the method of the invention. In other word, the method of the invention can perform typing of hundreds of thousands of SNPs from several ml of peripheral blood.

In the amplification step, a so-called multiplex polymerase chain reaction (multiplex PCR) is performed using genomic DNA prepared as a template DNA and a plurality of primer pairs for amplifying nucleotide sequences containing SNP sites to be genotyped. A preferred plurality of primer pairs are designed so as to be able to amplify 100 to 1500 bp DNA fragments flanking an SNP site. Each of these primers preferably comprises 17 to 25 nucleotides, more preferably, 18 to 22 nucleotides, respectively. These primers are designed so as to flank an SNP site to be genotyped, based on, for example, nucleotide sequence information accumulated in a database such as the GenBank.

The amplification step is performed by thermally denaturing template DNA (genomic DNA), followed by repetition of a cycle consisting of a thermal denaturation process to denature the template DNA, an annealing process to precisely anneal a plurality of primers, and an extension process to synthesize a DNA strand from the annealed primers. Finally, another extension step is performed to further extend the DNA strand. In addition, an appropriate temperature and time are preferably set separately for each process.

In such an amplification step, a preferred amount of a template DNA is 10 to 40 ng when, for example, nucleotide sequences of 100 regions are amplified using 100 pairs of primers. When the amount of a template DNA is less than 10 ng, amplifying all 100 regions would be difficult. In other words, with 10 ng or more of a template DNA, DNA fragments can be amplified in an amount sufficient to perform the typing step described later. When the amount of template DNA exceeds 40 ng, it becomes impractical to genotype hundreds of thousands of SNP sites because a large amount of genomic DNA is required.

In such an amplification step, a so-called hot start method is preferably applied. The hot start method is a technique in which the extension reaction with DNA polymerase is started when a reaction solution reaches a temperature high enough to prevent an annealing error and dimerization of primers. Examples of the hot start method include a method in which at least one kind of composition essential for PCR is added only when a reaction solution reaches a high temperature, a method which uses a wax barrier, and a method which uses a monoclonal antibody for DNA polymerase.

In the method using a wax barrier, first in a reaction container, solid wax divides an upper layer solution containing DNA polymerase and template DNA from a lower layer solution containing a primer and dNTP. Subsequently, PCR is allowed to proceed only after wax is melted by heating to a certain temperature so as to mix the upper and lower layer solutions.

In the method using a monoclonal antibody for DNA polymerase, DNA polymerase and a monoclonal antibody are bound to each other, and thus DNA polymerase is inactive until a reaction solution reaches a certain temperature. When the reaction solution is heated to a certain temperature (approximately 70° C. or more), the monoclonal antibody is irreversibly, thermally denatured and released from DNA polymerase. Thus, DNA polymerase is activated and PCR proceeds.

In any of these methods, the hot start method can prevent extension reaction from proceeding where annealing errors occur for each primer, or dimerization of primers to each other. Therefore, amplification of undesired DNA fragments can be prevented.

When the above-mentioned hot start method is applied in the amplification step, nucleotide sequences of 300 or more regions can be amplified simultaneously using 300 or more primer pairs. Hence, the method of the present invention enables typing of a greater number of SNPs, because application of the above hot start method allows amplification of a greater number of nucleotide sequences at one time.

2. Typing Step:

The typing step is a process to genotype a plurality of SNPs using DNA fragments amplified in the above-mentioned amplification step. In the method of the present invention, typing can be performed, for example, by applying a TaqMan PCR method or an Invader assay using the DNA fragments obtained in the amplification step.

The TaqMan PCR method utilizes PCR using a fluorescence-labeled allele-specific oligonucleotide (hereinafter, referred to as TaqMan probe), a template DNA containing an SNP to be genotyped, and Taq DNA polymerase (Livak, K. J. Genet. Anal. 14, 143 (1999); Morris T. et al., J. Clin. Microbiol. 34, 2933 (1996)). A TaqMan probe is designed based on SNP information, and which has a 5′ end labeled with fluorescent reporter dye R, such as FAM or VIC, and a 3′ end labeled with quencher Q (FIG. 1) at the same time. Since, in this condition, the quencher absorbs fluorescence energy, fluorescence from the TaqMan probe cannot be detected. Further, since the 3′ end of the TaqMan probe is phosphorylated, no extension reaction occurs from the TaqMan probe during PCR reaction (FIG. 1).

The following reactions occur when PCR is performed for a template DNA using the above described TaqMan probe, primers designed to amplify a region containing SNP site, and Taq DNA polymerase. First, the TaqMan probe hybridizes to a sequence specific to the template DNA (FIG. 2a), while extension reaction occurs from the PCR primer hybridizing to the template DNA (FIG. 2b). At this time, Taq DNA polymerase having 5′ nuclease activity cleaves nucleotides containing fluorescent reporter dye R of the TaqMan probe when extension reaction from PCR primers reaches the 5′ end of the TaqMan probe (FIG. 2c). When a nucleotide containing fluorescent reporter dye R is cleaved as described above, the fluorescent reporter dye R becomes unaffected by quencher Q, and emits a measurable fluorescence signal. Hence, detection of fluorescence of the fluorescent reporter dye R by a fluorescent detector enables confirmation of hybridization of the TaqMan probe and the template DNA.

For example, as shown in FIG. 3, an SNP site is supposed to be present at A in allele 1 (supposed to be allele 1) and that at G in allele 2 (supposed to be allele 2). While a TaqMan probe specific to allele 1 is labeled with FAM, a TaqMan probe specific to allele 2 is labeled with VIC (FIG. 3). The two types of TaqMan probe are added to PCR reagent, and then TaqMan PCR is performed for a template DNA containing SNP to be genotyped. PCR causes a nucleotide containing fluorescent reporter dye R to be released from a TaqMan probe having a nucleotide sequence complementary to SNP site to be genotyped, so that fluorescence is emitted. Then, fluorescent intensity of FAM and that of VIC are measured using a fluorescent detector.

As a result, SNP in the template DNA can be genotyped as a homozygote of allele 1 when strong fluorescence of FAM and almost no fluorescence of VIC are detected with a fluorescent detector. SNP in a template DNA can also be genotyped as a heterozygote of allele 1 and allele 2 when fluorescence of both FAM and VIC is detected with a fluorescent detector. Further, SNP in the template DNA can be genotyped as a homozygote of allele 2 when strong fluorescence of VIC and almost no fluorescence of FAM are detected with a fluorescent detector.

On the other hand, the Invader assay uses DNA comprising the SNP to be genotyped, two types of reporter probes specific to each allele of SNP to be genotyped, one type of Invader probe, and enzyme having special endonuclease activity to cleave by recognizing DNA structure (Livak, K. J. Biomol. Eng. 14, 143-149 (1999); Morris T. et al., J. Clin. Microbiol. 34, 2933 (1996); Lyamichev, V. et al., Science, 260, 778-783 (1993) and the like). In the Invader assay, SNP site can be genotyped by hybridization of an allele-specific oligonucleotide and DNA containing SNP to be genotyped. The Invader assay uses two types of unlabeled oligo and one type of fluorescence-labeled oligo. One of the two types of unlabeled oligo is called an allele probe.

An allele probe comprises a 3′ hybridizing region which hybridizes to a genomic DNA (template DNA) to form a complementary strand, and a 5′ region (called FLAP) which has a sequence unrelated to the sequence of a genomic DNA and does not hybridize to the genomic DNA. A nucleotide located at the 5′ end of the hybridizing region corresponds to an SNP (FIG. 4a). That is, an allele probe is provided with the hybridizing region which can form a complementary strand with a region on the 5′ side from the SNP site of genomic DNA (“A” in FIG. 4a), and Flap region which has been added to the 5′ side of a nucleotide (“T” in FIG. 4a) corresponding to the SNP. Here, the term “Flap” is an oligonucleotide having a sequence complementary to a given region of a FRET probe which is described later.

Another unlabeled oligo is called an Invader probe. The Invader probe is designed so as to complementarily hybridize in a direction from the SNP site (“A” in FIG. 4b) to the 3′ end of a genomic DNA (FIG. 4b). Here, a sequence (“N” in FIG. 4b) corresponding to an SNP site may be an arbitrary nucleotide. Thus, hybridization of a genomic DNA as a template and the above two probes causes one nucleotide (N) of the invader probe to invade into the SNP site (FIG. 4c).

A fluorescence-labeled oligo is a sequence totally independent from genomic DNA, and the sequence is a common sequence regardless of the type of SNP. This fluorescence-labeled oligo is called a FRET probe (fluorescence resonance energy transfer probe) (FIG. 5). A nucleotide (reporter, located at the 5′ terminus of FRET probe) is labeled with fluorescent dye (R), and quencher (Q) is bound upstream of the nucleotide. In this condition, no fluorescence can be detected with a fluorescent detector since the quencher absorbs fluorescence.

A certain region (region 1) from the 5′ end (reporter nucleotide) of FRET probe is designed to face a region (region 2) on the 3′ side of the region 1 so as to form a complementary sequence. Hence, in FRET probe, region 1 forms a complementary strand with region 2 (FIG. 5). Moreover, a region located further on the 3′ side of the region forming a complementary strand, that is, the 3′ terminal side of region 2 is designed so as to be able to form a complementary strand by hybridizing to Flap of an allele probe (FIG. 5).

The Invader assay uses Cleavase which is an enzyme (5′ nucleotidase) having special endonuclease activity to recognize and cleave a specific structure of DNA. Cleavase can cleave the 3′ side of an SNP position of an allele probe when a genomic DNA, allele probe and Invader probe overlap three fold at the SNP position. That is when three nucleotides overlap as shown in FIG. 4c, cleavase recognizes a portion at which the 5′ end is Flap-shaped, and cleaves the Flap portion. Therefore, cleavase recognizes this structure of an SNP site (FIG. 6a), the allele probe is cleaved at a nucleotide corresponding to the SNP site, and then the Flap portion is released (FIG. 6b).

Next, the Flap portion released from the allele probe binds complementarily to the FRET probe since it has a sequence complementary to that of the FRET probe (FIG. 6c). At this time, the SNP site of Flap invades into the complementary binding site of FRET itself. Again, cleavase recognizes the structure and cleaves a reporter nucleotide having fluorescent dye. The cleaved fluorescent dye becomes unaffected by the quencher and emits a measurable fluorescence signal (FIG. 6d). In addition, when a nucleotide corresponding to an SNP of allele probe does not match an SNP site, as shown in FIG. 7, the allele probe is not cleaved and Flap is not released since no specific DNA structure, which is specifically recognized by cleavase, is formed. Therefore, in this case almost all the fluorescent dye is still bound to the reporter nucleotide, and can emit any only low fluorescence signal.

Specifically, when an SNP site can be T or C, an Invader probe, an allele probe for T, and a FRET probe in which FAM has been bound to a reporter nucleotide corresponding to SNP is prepared. Separately, an Invader probe, an allele probe for C, and a FRET probe in which VIC has been bound to a reporter corresponding to SNP is prepared. All of them are mixed and the Invader assay is performed. An SNP site is then genotyped by detecting fluorescent intensity of FAM and that of VIC with a fluorescence detector. That is when strong fluorescence of FAM is detected but very low fluorescence of VIC is detected, the SNP can be genotyped as a homozygote (T/T). When fluorescence of both FAM and VIC can be detected, the SNP can be genotyped as a heterozygote (T/C). Further, when strong fluorescence of VIC is detected but very low fluorescence of FAM is detected, the SNP can be genotyped as a homozygote (T/T).

Now, a so-called SniPer method may be used in the typing step. The SniPer method is based on a technique called RCA (rolling circle amplification) method. In the SniPer method, DNA polymerase sequentially synthesizes a complementary strand DNA while migrating on a circular single stranded DNA as a template. According to the method, SNP can be genotyped by measuring the presence or absence of coloring reaction due to DNA amplification (Lizardi, P. M. et al., Nature Genet., 19, 225-232 (1998); Piated, A. S. et al., Nature Biotech., 16, 359-363 (1998)).

Particularly in the typing step, card 1, as shown in FIG. 8a, is preferably used when any of the methods described above is employed. Card 1 comprises numerous wells 2, arranged in a matrix on a primary surface thereof, wall surfaces 3 which are so formed as to project around each well 2, and a plurality of grooves 4 which are formed between adjacent wells 2. An example of a card 1 that can be used is, but is not limited to, a card 1 comprising a total of 384 wells 2 (24 columns×16 lines) formed thereon.

When the typing step is performed using card 1, first the DNA fragment obtained in the above-mentioned amplification step is dispensed into wells 2, in the form of PCR reaction solution. Then, the dispensed PCR reaction solution is dried up, so that the dried DNA fragments remain on the bottom surfaces of wells 2.

Subsequently, as shown in FIG. 8b, a reagent necessary for the typing step, for example, a reagent necessary for the Invader assay is dispensed into wells 2. At this time, the reagent necessary for the typing step is dispensed onto the upper portion of a wall surface 3 in a volume exceeding that of well 2 but not such that it overflows, due to surface tension. Further, the DNA fragment present in a dried state in well 2 is dissolved in the reagent dispensed into wells 2, necessary for the typing step. When a reagent necessary for the typing step is dispensed into wells 2, a pipette preferably used herein is a non-contact dispensing equipment which is capable of simultaneously dispensing a solution into a plurality of wells 2 arranged on card 1. Since a non-contact dispensing equipment does not contact with the inside of wells 2, such a pipette can prevent contamination from occurring between a plurality of cards 1.

Next as shown in FIG. 8c, plastic plate 5 which is large enough to cover the primary surface of card 1 is overlayed on the primary surface of card 1. Accordingly, the reagent dispensed into wells 2 flows out toward the outside of wells 2. Most of the reagent that has flowed out remains within grooves 4.

Then, card 1 with plastic plate 5 overlayed on the primary surface thereof is ultrasonically treated with a ultrasonic welding equipment, so that plastic plate 5 and card 1 are welded. As specifically shown in FIG. 8d, the upper surfaces of the wall surfaces 3 and the plastic plate 5 are in contact with each other, and welding can be performed at the contacting area.

As described above, the use of card 1 in the typing step enables prevention of foaming within wells 2 and prevents the reagent dispensed within wells 2 from flowing out even when card 1 is subjected to heat treatment. Therefore, card 1 employed in the typing step can improve detection sensitivity for a fluorescent dye or the like.

Further, use of card 1 provided with well 2 whose volume is 0.6 ml can largely decrease the volume of reagent necessary for the typing step, so as to largely reduce the cost required for SNP typing.

Furthermore, when the typing step is performed using card 1, heat treatment or the like included in the typing step is preferably performed using a thermostat water bath. By using a thermostat water bath, the typing step can be performed using numerous cards 1 simultaneously. For example, since performing the typing step simultaneously using numerous cards 1 is difficult when a thermal cycler is used, a thermostat water bath is preferably used.

3. Effect of the Method of the Present Invention

According to the method of the present invention, a target SNP can be genotyped by amplifying in the amplification step genomic DNA collected or extracted, and using the amplified DNA fragments. Therefore, the method enables typing of several hundreds of thousands of SNP sites even with a small amount of genomic DNA. For example, when 100,000 SNP sites are genotyped, approximately 10 μg of genomic DNA would be required according to an estimation that approximately 0.1 ng of genomic DNA is required per SNP site. Approximately 10 μg of genomic DNA can be extracted from 1.25 ml of peripheral blood collected from a human.

On the other hand, since SNP typing using genomic DNA it self by the Invader assay or the like requires several tens ng of genomic DNA per SNP site, several mg of genomic DNA must be prepared to genotype 100,000 SNP sites. To obtain several mg of genomic DNA, 500 ml or more peripheral blood must be collected. However, it is actually impossible to prepare genomic DNA in such a volume.

### BRIEF DESCRIPTION OF THE DRAWINGS

FIG. 1 schematically shows TaqMan probes.

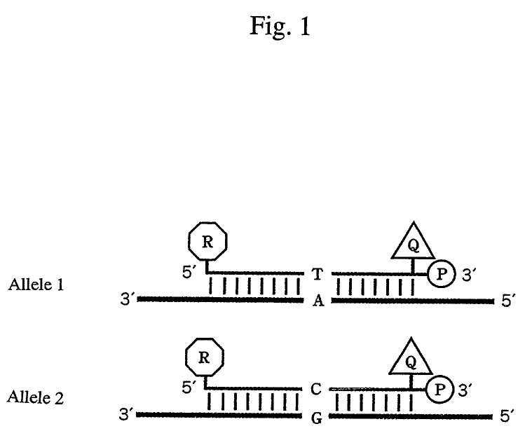

FIG. 2 shows the outline of each steps composing the TaqMan PCR method.

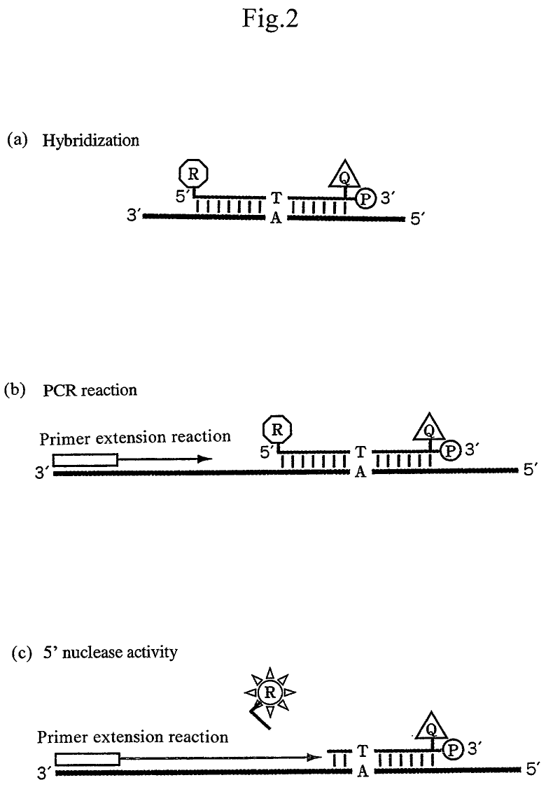

FIG. 3 schematically shows fluorescence-labeled TaqMan probes.

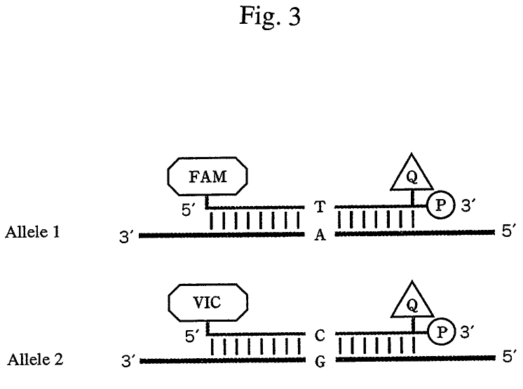

FIG. 4 schematically shows the Invader assay.

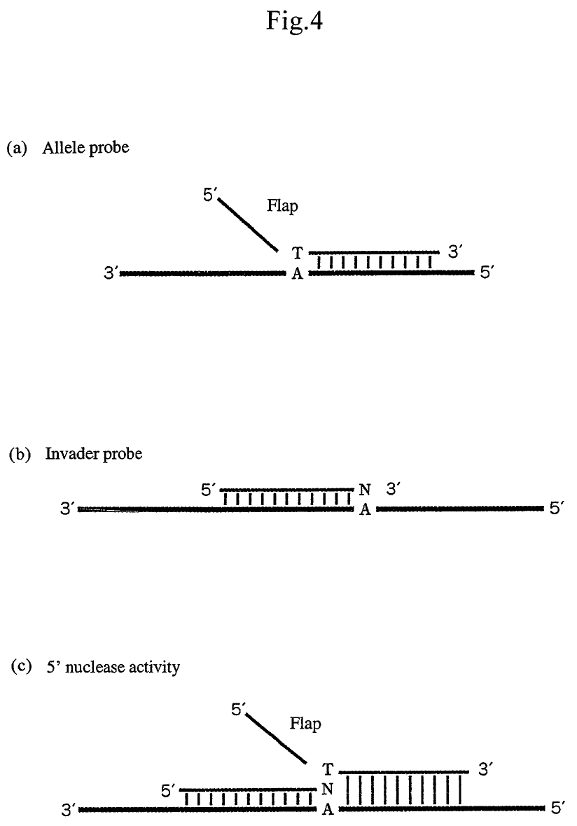

FIG. 5 schematically shows a FRET probe.

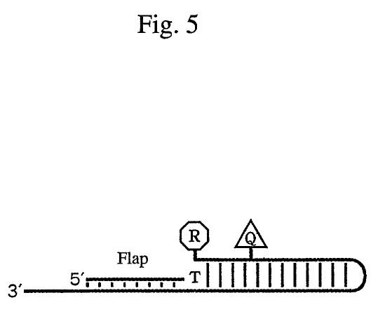

FIG. 6 schematically shows the Invader assay.

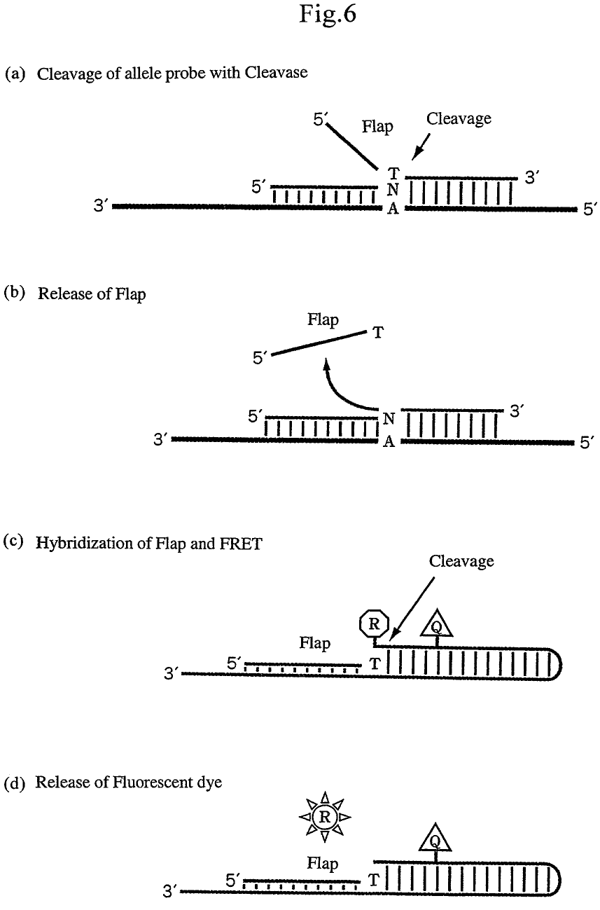

FIG. 7 schematically shows a probe which does not match an allele.

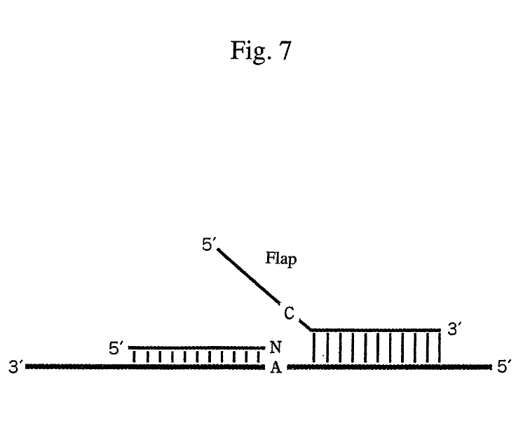

FIG. 8 shows a partial cross-sectional view of a card used in the typing step.

FIG. 9 is a characteristic figure showing the result of typing SNP No. 1.

FIG. 10 is a characteristic figure showing the result of typing by the Invader assay directly using genomic DNA.

FIG. 11 is a flowchart of the method for typing described in example 4.

FIG. 12 is a characteristic figure showing the result of detecting a signal intensity of VIC/ROX and FAM/ROX in example 4.

### EXAMPLE

Now the present invention will be described more specifically, but the technical scope of the invention is not limited by the following examples.

### Example 1

### Preparation of Genomic DNA

Leukocytes were isolated from the peripheral blood collected from a subject who had given informed consent, and genomic DNA was extracted therefrom. Genomic DNA was extracted according to Laboratory Manual for Genomic Analysis (Yusuke Nakamura ed., Springer-Verlag Tokyo) as described below. 10 ml of the blood was transferred to a 50 ml Falcon tube, and then centrifuged at 3,000 rpm for 5 min at room temperature. The supernatant (serum) was discarded with a pipette, 30 ml of RBC lysis buffer (10 mM NH₄HCO₃, 144 mM NH₄Cl) was added thereto. After mixing to disperse the precipitate well, the mixture was allowed to stand at room temperature for 20 min. Subsequently, centrifugation was performed at 3,000 rpm for 5 min at room temperature, the supernatant (serum) was discarded with a pipette, thereby obtaining a leukocyte pellet. 30 ml of RBC lysis buffer was added to the pellet, and then a similar procedure was performed twice. 4 ml of Proteinase K buffer (50 mM Tris-HCl(pH 7.4), 100 mM NaCl, 1 mM EDTA(pH 8.0)), 200 μl of 10% SDS, 200 μl of 10 mg/ml Proteinase K were added to the leukocyte pellet. The mixture was mixed by inverting, and allowed to stand at 37° C. overnight. 4 ml of phenol was added to the mixture, and then mixed by slowly inverting for 4 hours with a rotator (Rotator T-50, Taitec). Centrifugation was performed at 3,000 rpm for 10 min at room temperature, and the upper layer was collected in a new tube. 4 ml of phenol-chloroform-isoamyl alcohol (volume ratio 25:24:1) was added to the product, followed by two hours of similar mixing by inverting and centrifugation. The upper layer was collected in a new tube, and 4 ml of chloroform-isoamyl alcohol (volume ratio 24:1) was added thereto. Then the product was mixed by inverting similarly for 30 min, followed by centrifugation. The upper layer was collected in a new tube, and 400 μl of 8 M ammonium acetate and 4 ml of isopropanol were added thereto, followed by mixing by inverting. White, filamentous precipitate (DNA) was collected in a 2 ml tube, 1 ml of 70% ethanol was added thereto, followed by mixing by inverting. DNA was collected in a new 2 ml tube and then air-dried. DNA was dissolved in 500 μl of TE solution (10 mM Tris-HCl (pH 7.4), 1 mM EDTA (pH7.4)), thereby preparing a genomic DNA sample.

### Example 2

### Amplification of Genomic DNA

PCR was performed with a 50 μl system using 40 ng of the genomic DNA obtained in Example 1. A reaction solution contains 200 types of primer (50 pmol 100 pairs, SEQ ID NOS: 1 to 200), 10 units of EX-TaqDNA polymerase (Takara Shuzo), and 0.55 μg of TaqStart (CLONTECH Laboratories). TaqStart is an antibody for EX-TaqDNA polymerase. The hot start method can be performed by adding TaqStart to the reaction solution.

PCR was performed with GeneAmp PCR system 9700 (Applied Biosystems). DNA was denatured at 94° C. for 2 min, a cycle consisting of a denaturation process at 94° C. for 15 sec, an annealing process at 60° C. at 45 sec, and then an extension process at 72° C. for 3 min was repeated 35 times, followed by extension at 72° C. for 3 min.

As shown in Table 1, a plurality of DNA fragments containing SNP Identification Nos. (“SNP ID” in Table 1) 1 to 100 can be amplified simultaneously.

TABLE 1

| SNP ID | Forward primer | Reverse primer | SNP name |
|---|---|---|---|
| 1 | SEQ ID NO 1 | SEQ ID NO 2 | AC000353.27_20000214_5_24737 |
| 2 | SEQ ID NO 3 | SEQ ID NO 4 | AC000388.1_19970529_9_37703 |
| 3 | SEQ ID NO 5 | SEQ ID NO 6 | AC001643.1_19970529_3_6293 |
| 4 | SEQ ID NO 7 | SEQ ID NO 8 | AC002237.1_19970606_1_1204 |
| 5 | SEQ ID NO 9 | SEQ ID NO 10 | AC002319.1_19980203_3_29222 |
| 6 | SEQ ID NO 11 | SEQ ID NO 12 | AC002364.1_19981204_2_117944 |
| 7 | SEQ ID NO 13 | SEQ ID NO 14 | AC003005.1_19971022_1_2731 |
| 8 | SEQ ID NO 15 | SEQ ID NO 16 | AC003005.1_19971022_3_5667 |
| 9 | SEQ ID NO 17 | SEQ ID NO 18 | AC003689.1_19981121_2_45471 |
| 10 | SEQ ID NO 19 | SEQ ID NO 20 | AF066064.1_19980603_1_563 |
| 11 | SEQ ID NO 21 | SEQ ID NO 22 | AF077374.1_19990202_1_1708 |
| 12 | SEQ ID NO 23 | SEQ ID NO 24 | AF157101.1_19990624_1_618 |
| 13 | SEQ ID NO 25 | SEQ ID NO 26 | AF196968.1_19991109_1_6368 |
| 14 | SEQ ID NO 27 | SEQ ID NO 28 | AJ009610.1_19990104_4_26810 |
| 15 | SEQ ID NO 29 | SEQ ID NO 30 | AJ011772.1_19981005_2_903 |
| 16 | SEQ ID NO 31 | SEQ ID NO 32 | AJ011931.1_19981110_5_23638 |
| 17 | SEQ ID NO 33 | SEQ ID NO 34 | AJ229043.1_19990122_1_3475 |
| 18 | SEQ ID NO 35 | SEQ ID NO 36 | AL008633.1_19971029_1_33923 |
| 19 | SEQ ID NO 37 | SEQ ID NO 38 | AL008634.1_19981109_13_92880 |
| 20 | SEQ ID NO 39 | SEQ ID NO 40 | AL008634.1_19981109_13_93343 |
| 21 | SEQ ID NO 41 | SEQ ID NO 42 | AL008634.1_19981109_14_95554 |
| 22 | SEQ ID NO 43 | SEQ ID NO 44 | AL008638.1_19981123_4_52385 |
| 23 | SEQ ID NO 45 | SEQ ID NO 46 | AL008730.1_19980204_2_66080 |
| 24 | SEQ ID NO 47 | SEQ ID NO 48 | AL008733.10_19991225_1_5608 |
| 25 | SEQ ID NO 49 | SEQ ID NO 50 | AL008734.10_19990610_1_7867 |
| 26 | SEQ ID NO 51 | SEQ ID NO 52 | AL021917.1_19980721_19_77217 |
| 27 | SEQ ID NO 53 | SEQ ID NO 54 | AL021937.1_19990303_93_124364 |
| 28 | SEQ ID NO 55 | SEQ ID NO 56 | AL022721.1_19990324_13_73842 |
| 29 | SEQ ID NO 57 | SEQ ID NO 58 | AL023279.1_19990305_3_69539 |
| 30 | SEQ ID NO 59 | SEQ ID NO 60 | AL049557.19_73359 |
| 31 | SEQ ID NO 61 | SEQ ID NO 62 | AL049569.13_164971 |
| 32 | SEQ ID NO 63 | SEQ ID NO 64 | AL049569.13_61322 |
| 33 | SEQ ID NO 65 | SEQ ID NO 66 | AL049569.13_61680 |
| 34 | SEQ ID NO 67 | SEQ ID NO 68 | AL049569.13_61971 |
| 35 | SEQ ID NO 69 | SEQ ID NO 70 | AL049569.13_62026 |
| 36 | SEQ ID NO 71 | SEQ ID NO 72 | AL049569.13_87106 |
| 37 | SEQ ID NO 73 | SEQ ID NO 74 | AL049569.13_87279 |
| 38 | SEQ ID NO 75 | SEQ ID NO 76 | AL049569.13_88461 |
| 39 | SEQ ID NO 77 | SEQ ID NO 78 | AL049569.13_88502 |
| 40 | SEQ ID NO 79 | SEQ ID NO 80 | AL049575.7_10509 |
| 41 | SEQ ID NO 81 | SEQ ID NO 82 | AL049611.24_75054 |
| 42 | SEQ ID NO 83 | SEQ ID NO 84 | AL049611.24_75895 |
| 43 | SEQ ID NO 85 | SEQ ID NO 86 | AL049612.11_45784 |
| 44 | SEQ ID NO 87 | SEQ ID NO 88 | AL049649.4_93434 |
| 45 | SEQ ID NO 89 | SEQ ID NO 90 | AL049649.4_93918 |
| 46 | SEQ ID NO 91 | SEQ ID NO 92 | AL049650.8_62150 |
| 47 | SEQ ID NO 93 | SEQ ID NO 94 | AL049691.17_64637 |
| 48 | SEQ ID NO 95 | SEQ ID NO 96 | AL049694.9_4336 |
| 49 | SEQ ID NO 97 | SEQ ID NO 98 | AL049698.3_3216 |
| 50 | SEQ ID NO 99 | SEQ ID NO 100 | AL049698.3_3822 |
| 51 | SEQ ID NO 101 | SEQ ID NO 102 | AL049758.11_67143 |
| 52 | SEQ ID NO 103 | SEQ ID NO 104 | AL049758.11_79044 |
| 53 | SEQ ID NO 105 | SEQ ID NO 106 | AL049759.10_111608 |
| 54 | SEQ ID NO 107 | SEQ ID NO 108 | AL049795.20_113584 |
| 55 | SEQ ID NO 109 | SEQ ID NO 110 | AL049829.2_138544 |
| 56 | SEQ ID NO 111 | SEQ ID NO 112 | AL049829.2_161140 |
| 57 | SEQ ID NO 113 | SEQ ID NO 114 | AL049843.18_49141 |
| 58 | SEQ ID NO 115 | SEQ ID NO 116 | AL096766.12_13162 |
| 59 | SEQ ID NO 117 | SEQ ID NO 118 | AP000065.1_58129 |
| 60 | SEQ ID NO 119 | SEQ ID NO 120 | AP000168.1_56285 |
| 61 | SEQ ID NO 121 | SEQ ID NO 122 | AP000171.1_87106 |
| 62 | SEQ ID NO 123 | SEQ ID NO 124 | AP000347.1_81990 |
| 63 | SEQ ID NO 125 | SEQ ID NO 126 | AP000349.1_19017 |
| 64 | SEQ ID NO 127 | SEQ ID NO 128 | AP000350.1_10554 |
| 65 | SEQ ID NO 129 | SEQ ID NO 130 | AP000350.1_10756 |
| 66 | SEQ ID NO 131 | SEQ ID NO 132 | AP000350.1_11294 |
| 67 | SEQ ID NO 133 | SEQ ID NO 134 | AP000350.1_31581 |
| 68 | SEQ ID NO 135 | SEQ ID NO 136 | AP000352.1_63635 |
| 69 | SEQ ID NO 137 | SEQ ID NO 138 | AP000353.1_86203 |
| 70 | SEQ ID NO 139 | SEQ ID NO 140 | AP000355.1_132012 |
| 71 | SEQ ID NO 141 | SEQ ID NO 142 | AP000493.1_129114 |
| 72 | SEQ ID NO 143 | SEQ ID NO 144 | AP000495.1_60416 |
| 73 | SEQ ID NO 145 | SEQ ID NO 146 | AP000500.1_113211 |
| 74 | SEQ ID NO 147 | SEQ ID NO 148 | AP000500.1_113401 |
| 75 | SEQ ID NO 149 | SEQ ID NO 150 | AP000500.1_194483 |
| 76 | SEQ ID NO 151 | SEQ ID NO 152 | AP000500.1_25277 |
| 77 | SEQ ID NO 153 | SEQ ID NO 154 | AP000501.1_37357 |
| 78 | SEQ ID NO 155 | SEQ ID NO 156 | AP000501.1_99530 |
| 79 | SEQ ID NO 157 | SEQ ID NO 158 | AP001041.1_6501 |
| 80 | SEQ ID NO 159 | SEQ ID NO 160 | AP001041.1_6582 |
| 81 | SEQ ID NO 161 | SEQ ID NO 162 | AP001054.1_35804 |
| 82 | SEQ ID NO 163 | SEQ ID NO 164 | AP001054.1_36083 |
| 83 | SEQ ID NO 165 | SEQ ID NO 166 | AP001054.1_36142 |
| 84 | SEQ ID NO 167 | SEQ ID NO 168 | AP001101.1_12400 |
| 85 | SEQ ID NO 169 | SEQ ID NO 170 | D42052.1_7718 |
| 86 | SEQ ID NO 171 | SEQ ID NO 172 | D50561.1_1218 |
| 87 | SEQ ID NO 173 | SEQ ID NO 174 | D50561.1_564 |
| 88 | SEQ ID NO 175 | SEQ ID NO 176 | NT_002717.1_29435 |
| 89 | SEQ ID NO 177 | SEQ ID NO 178 | U07563.1_68521 |
| 90 | SEQ ID NO 179 | SEQ ID NO 180 | X56832.1_2826 |
| 91 | SEQ ID NO 181 | SEQ ID NO 182 | X69299.1_1633 |
| 92 | SEQ ID NO 183 | SEQ ID NO 184 | X74107.1_29168 |
| 93 | SEQ ID NO 185 | SEQ ID NO 186 | X74107.1_29545 |
| 94 | SEQ ID NO 187 | SEQ ID NO 188 | X78901.1_1934 |
| 95 | SEQ ID NO 189 | SEQ ID NO 190 | X87344.1_115764 |
| 96 | SEQ ID NO 191 | SEQ ID NO 192 | X91863.1_2477 |
| 97 | SEQ ID NO 193 | SEQ ID NO 194 | Y08378.1_1560 |
| 98 | SEQ ID NO 195 | SEQ ID NO 196 | Y12852.1_4439 |
| 99 | SEQ ID NO 197 | SEQ ID NO 198 | Y16792.1_4230 |
| 100 | SEQ ID NO 199 | SEQ ID NO 200 | Z54246.1_8005 |

### Example 3

### Typing by Invader Assay

The sample obtained by PCR as described in Example 2 was apportioned, 0.2 μl each, to 100 tubes and then typing was performed for 100 types of SNPs using an Invader assay kit (Third Wave Technology). That is, 0.5 μl of the sample was added to the kit containing 0.5 μl of signal buffer, 0.5 μl of FRET probe, 0.5 μl of structure-specific deoxyribonuclease, and 1 μl of allele-specific probe. The reaction volume was prepared to be 10 μl. FRET probes were labeled with different fluorescent dyes (FAM and VIC). Two types of FRET probes differing in their Flap complementary sequences were used. A pair of probes has Flap portions corresponding to two types of FRET probes. Next, the reaction solution was incubated at 95° C. for 5 min, and then 63° C. for 15 min using ABI7700 (Applied Biosystems). Fluorescence emitted during incubation was detected using the device.

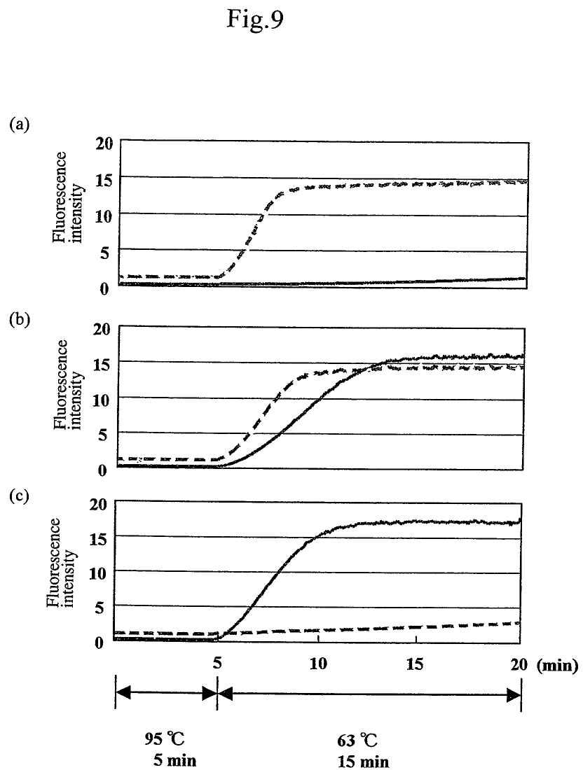

FIGS. 9a, b and c show respectively the results of typing three different samples using SNP ID NO: 1 probe. In FIG. 9, a continuous line denotes fluorescence of FAM, and a broken line denotes that of VIC. As shown in FIG. 9a, only the fluorescence of VIC was elevated for this sample. This result suggested that both alleles of nucleotides (SNP ID NO: 1) in this sample corresponded to a specific probe having a Flap complementary to FRET probe labeled with VIC, that is, the sample was homozygous. The result for the sample in FIG. 9b suggested that one of the alleles corresponded to a specific probe having a Flap complementary to FAM-labeled FRET probe, and the other allele corresponded to a specific probe having a Flap complementary to VIC-labeled FRET probe, that is, this sample was heterozygous. Further, the sample in FIG. 9c was shown to be homozygous corresponding to a specific probe having a Flap complementary to FAM-labeled FRET probe.

Moreover, fluorescence was detected for 98% of SNPs (SNP ID NOS: 1 to 100). The result suggested that with a very small amount of genomic DNA, 0.4 ng per SNP, typing was possible by the method of the present invention.

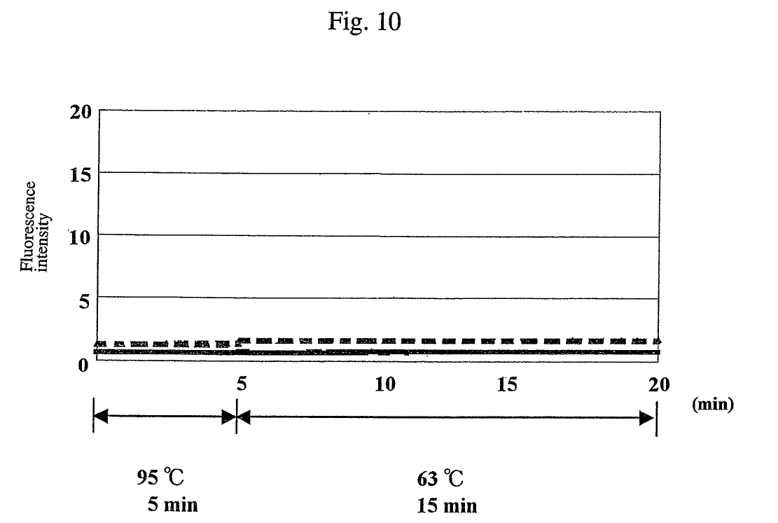

When the Invader assay was directly performed using 0.4 ng of genomic DNA, no fluorescence was detected and typing was impossible as shown in FIG. 10. Probably, this was due to the amount of DNA (0.4 ng) used for assay being insufficient to obtain fluorescence required for detection.

### Example 4

### Improved Method

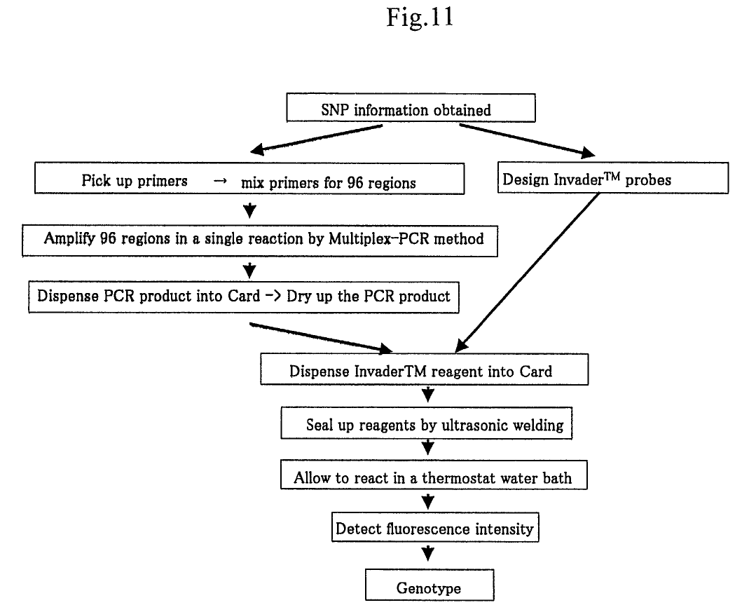

A typing system using a smaller quantity of genomic DNA was studied by improving the typing system described in Example 3. In this example, a 96-well PCR plate was used and 96 DNA fragments were amplified in each well with a single amplification reaction. In addition, the flow chart of the typing system performed in this Example 4 is shown in FIG. 11.

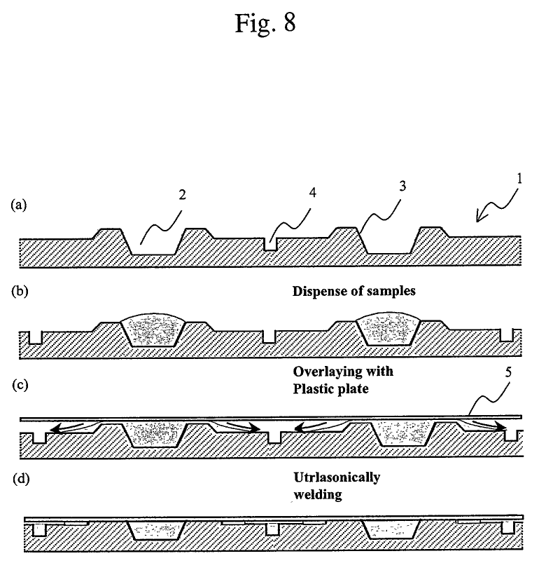

The PCR product obtained in Example 2 was diluted and then transferred into a 384 deep well. Then 0.8 μl of the PCR product was dispensed into each well (volume: 0.6 μl) of a card shown in FIG. 8a. An automatic liquid handling system, Tango (Robbins), was used for dispensing. The PCR product was dispensed using an automatic liquid handling system Tango from a single plate having 384 deep wells to 96 cards. The automatic liquid handling system Tango is capable of simultaneously dispensing 384 samples, that is, capable of dispensing into 8 cards in a single operation. Thereafter, the dispensed PCR product was naturally dried at room temperature.

Next, 0.03 μl of signal buffer, 0.03 μl of FRET probe, 0.03 μl of structure specific deoxyribonuclease and 0.06 μl of allele specific probe contained in an Invader assay kit (Third Wave Technology) were dispensed into wells. These solutions were dispensed using a non-contact dispensing workstation PixSys4200 (Cartesian Technologies). The non-contact dispensing workstation PixSys4200 is capable of simultaneously dispensing the above solutions into 8 cards, each having 384 wells.

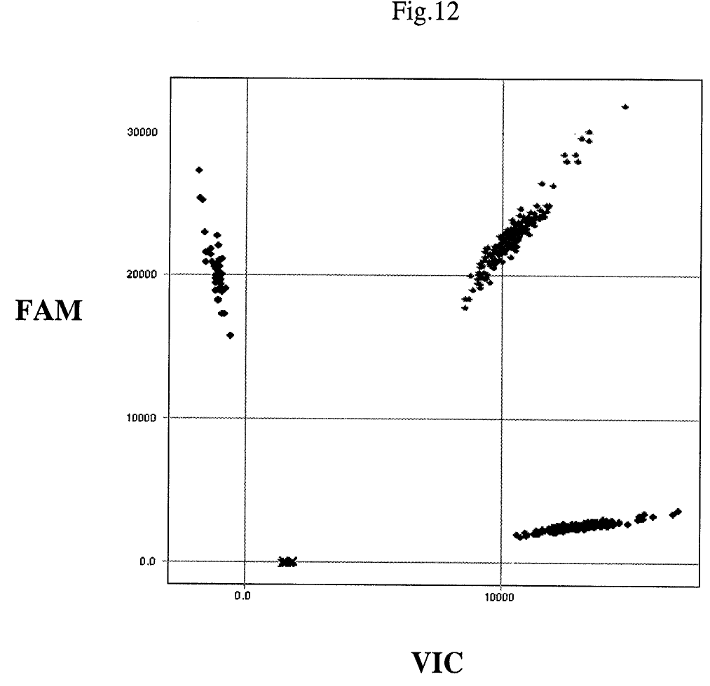

Subsequently, a plastic plate was overlayed on the card, and then ultrasonic welding using ultrasonic welding equipment (Branson) was performed. Thus, 96 cards, each having 384 wells, could be prepared. Then, incubation was performed using a thermobath (TAITEC) at 95° C. for 5 min, followed by 63° C. for 60 min. After incubation, fluorescence emitted from each well of the cards was detected using a fluorescence detector ABI 7900(Applied Biosystems), and typing was performed. FIG. 12 shows the result detecting a signal intensity of VIC/ROX and FAM/ROX. In FIG. 12, the horizontal axis shows the signal intensity of VIC/ROX; the vertical axis shows the signal intensity of FAM/ROX. The bottom right cluster of spots indicates a strong VIC/ROX signal and a weak FAM/ROX signal; the samples in this cluster are judged to be homozygous for allele 1. Similarly, samples indicated by the top right cluster of spots are heterozygous, and samples indicated by the top left cluster of spots are homozygous for allele 2.

Although only 0.1 ng genomic DNA was used as template, alleles could be discriminated clearly.

According to Example 4, as shown in FIG. 12, alleles could be discriminated clearly and fluorescence could be detected with high sensitivity even when 0.1 ng of the genomic DNA was subjected to a single typing, thereby allowing accurate typing. Thus, genomic DNA to be subjected to a single PCR reaction in an amplification step would be approximately 10 ng. Therefore according to Example 4, with genomic DNA in a volume approximately ¼ of that used in the method of Example 3, typing can be performed.

Effect of the Invention

As described above in detail, the method for SNP typing according to the present invention can type several hundreds of thousands of SNP sites using a very small amount of genomic DNA. Hence, the method for SNP typing according to the present invention enables typing of several hundreds of thousands of SNP sites using very small amount of genomic DNA at low cost and in a short period of time.

Sequence Listing Free Text

SEQ ID NOS: 1 to 200 are synthetic primers.

What is claimed is:

1. A method for SNP typing which comprises the steps of:
simultaneously amplifying a plurality of nucleotide sequences comprising at least one or more sites of single nucleotide polymorphism using genomic DNA and a plurality of primer pairs; and
typing for distinguishing the site(s) of single nucleotide polymorphism of nucleotides contained in a plurality of nucleotide sequences amplified in the above amplification step using the amplified nucleotide sequences.

2. The method for SNP typing according to claim 1, wherein said step of amplifying employs the polymerase chain reaction using a hot start method.

3. The method for SNP typing according to claim 1, wherein said step of amplifying employs 50 pairs or more primers.

4. The method for SNP typing according to claim 1, wherein said step of typing employs an Invader assay or a TaqMan PCR method.

5. The method for SNP typing according to claim 1, wherein amplification is carried out from 10 ng to 40 ng of genomic DNA.


SEQUENCE LISTING (200 sequences)

SEQ ID NO: 1 - DNA, 20 bp, Artificial Sequence
  Description of Artificial Sequence Forward Primer for SNP ID 1
ccagcaggacttggtgacag

SEQ ID NO: 2 - DNA, 21 bp, Artificial Sequence
  Description of Artificial Sequence Reverse Primer for SNP ID 1
gcaagaagcagccagatcaag

SEQ ID NO: 3 - DNA, 20 bp, Artificial Sequence
  Description of Artificial Sequence Forward Primer for SNP ID 2
cagccacccactcagtcttg

SEQ ID NO: 4 - DNA, 20 bp, Artificial Sequence
  Description of Artificial Sequence Reverse Primer for SNP ID 2
aggtcctggctctgcgtaac

SEQ ID NO: 5 - DNA, 22 bp, Artificial Sequence
  Description of Artificial Sequence Forward Primer for SNP ID 3
gcttgagactcaccctctgatg

SEQ ID NO: 6 - DNA, 20 bp, Artificial Sequence
  Description of Artificial Sequence Reverse Primer for SNP ID 3
gtcccgacttgaaggtccac

SEQ ID NO: 7 - DNA, 22 bp, Artificial Sequence
  Description of Artificial Sequence Forward Primer for SNP ID 4
tctgccaagcagaaacctagag

SEQ ID NO: 8 - DNA, 19 bp, Artificial Sequence
  Description of Artificial Sequence Reverse Primer for SNP ID 4
ggcaccttgagaggaatgc

SEQ ID NO: 9 - DNA, 19 bp, Artificial Sequence
  Description of Artificial Sequence Forward Primer for SNP ID 5
ctttccgacaacgagagcg

SEQ ID NO: 10 - DNA, 20 bp, Artificial Sequence
  Description of Artificial Sequence Reverse Primer for SNP ID 5
cacctggactctgcatcctg

SEQ ID NO: 11 - DNA, 19 bp, Artificial Sequence
  Description of Artificial Sequence Forward Primer for SNP ID 6
aggccgtgagggaatgatg

SEQ ID NO: 12 - DNA, 20 bp, Artificial Sequence
  Description of Artificial Sequence Reverse Primer for SNP ID 6
gggtgtctagcatggtgctg

SEQ ID NO: 13 - DNA, 21 bp, Artificial Sequence
  Description of Artificial Sequence Forward Primer for SNP ID 7
ttcagcatagctccagaaggc

SEQ ID NO: 14 - DNA, 22 bp, Artificial Sequence
  Description of Artificial Sequence Reverse Primer for SNP ID 7
tttggacccttgtcctaacaac

SEQ ID NO: 15 - DNA, 23 bp, Artificial Sequence
  Description of Artificial Sequence Forward Primer for SNP ID 8
tggctcactaaatgcactaccac

SEQ ID NO: 16 - DNA, 20 bp, Artificial Sequence
  Description of Artificial Sequence Reverse Primer for SNP ID 8
acctggaggtgaagcgagac

SEQ ID NO: 17 - DNA, 20 bp, Artificial Sequence
  Description of Artificial Sequence Forward Primer for SNP ID 9
accacaggctccaggaagtg

SEQ ID NO: 18 - DNA, 19 bp, Artificial Sequence
  Description of Artificial Sequence Reverse Primer for SNP ID 9
tgcgtttgcactggtaggc

SEQ ID NO: 19 - DNA, 20 bp, Artificial Sequence
  Description of Artificial Sequence Forward Primer for SNP ID 10
cactcccaccaccatcactg

SEQ ID NO: 20 - DNA, 20 bp, Artificial Sequence
  Description of Artificial Sequence Reverse Primer for SNP ID 10
gctcacggaactcgaagacg

SEQ ID NO: 21 - DNA, 22 bp, Artificial Sequence
  Description of Artificial Sequence Forward Primer for SNP ID 11
caggtgacatcactgtcagagc

SEQ ID NO: 22 - DNA, 20 bp, Artificial Sequence
  Description of Artificial Sequence Reverse Primer for SNP ID 11
acctgctgctgttgaagctg

SEQ ID NO: 23 - DNA, 23 bp, Artificial Sequence
  Description of Artificial Sequence Forward Primer for SNP ID 12
cgttggaagcctgtactccttag

SEQ ID NO: 24 - DNA, 20 bp, Artificial Sequence
  Description of Artificial Sequence Reverse Primer for SNP ID 12
aggagagctcacccgaagtg

SEQ ID NO: 25 - DNA, 20 bp, Artificial Sequence
  Description of Artificial Sequence Forward Primer for SNP ID 13
ggcacctctccaggattgtg

SEQ ID NO: 26 - DNA, 20 bp, Artificial Sequence
  Description of Artificial Sequence Reverse Primer for SNP ID 13
gaagccagggcaagtcattg

SEQ ID NO: 27 - DNA, 20 bp, Artificial Sequence
  Description of Artificial Sequence Forward Primer for SNP ID 14
cccaaggcccactgtgttac

SEQ ID NO: 28 - DNA, 20 bp, Artificial Sequence
  Description of Artificial Sequence Reverse Primer for SNP ID 14
cctggtgccaagtggtcaag

SEQ ID NO: 29 - DNA, 20 bp, Artificial Sequence
  Description of Artificial Sequence Forward Primer for SNP ID 15
gagcattgccctcctcactg

SEQ ID NO: 30 - DNA, 22 bp, Artificial Sequence
  Description of Artificial Sequence Reverse Primer for SNP ID 15
gtgccacaattgatatgaccag

SEQ ID NO: 31 - DNA, 20 bp, Artificial Sequence
  Description of Artificial Sequence Forward Primer for SNP ID 16
gcctctacctttaccgtccg

SEQ ID NO: 32 - DNA, 20 bp, Artificial Sequence
  Description of Artificial Sequence Reverse Primer for SNP ID 16
gccacctccctgtcttcatc

SEQ ID NO: 33 - DNA, 21 bp, Artificial Sequence
  Description of Artificial Sequence Forward Primer for SNP ID 17
cagcttcaggccaaatgtatg

SEQ ID NO: 34 - DNA, 20 bp, Artificial Sequence
  Description of Artificial Sequence Reverse Primer for SNP ID 17
tcacacctcctcctccattg

SEQ ID NO: 35 - DNA, 20 bp, Artificial Sequence
  Description of Artificial Sequence Forward Primer for SNP ID 18
tccaccctgatcaagtccag

SEQ ID NO: 36 - DNA, 19 bp, Artificial Sequence
  Description of Artificial Sequence Reverse Primer for SNP ID 18
gcatgggtgcactgttgac

SEQ ID NO: 37 - DNA, 22 bp, Artificial Sequence
  Description of Artificial Sequence Forward Primer for SNP ID 19
aaattaaggcacaggcagtgag

SEQ ID NO: 38 - DNA, 20 bp, Artificial Sequence
  Description of Artificial Sequence Reverse Primer for SNP ID 19
gtcctctgctttgctcaggc

SEQ ID NO: 39 - DNA, 22 bp, Artificial Sequence
  Description of Artificial Sequence Forward Primer for SNP ID 20
aaattaaggcacaggcagtgag

SEQ ID NO: 40 - DNA, 20 bp, Artificial Sequence
  Description of Artificial Sequence Reverse Primer for SNP ID 20
gtcctctgctttgctcaggc

SEQ ID NO: 41 - DNA, 24 bp, Artificial Sequence
  Description of Artificial Sequence Forward Primer for SNP ID 21
tctatgtgggtaggatctccagac

SEQ ID NO: 42 - DNA, 21 bp, Artificial Sequence
  Description of Artificial Sequence Reverse Primer for SNP ID 21
tcgaaacagaagatgtggctg

SEQ ID NO: 43 - DNA, 20 bp, Artificial Sequence
  Description of Artificial Sequence Forward Primer for SNP ID 22
cagcagcaacaacaaccgtc

SEQ ID NO: 44 - DNA, 24 bp, Artificial Sequence
  Description of Artificial Sequence Reverse Primer for SNP ID 22
cccaagtgtggtaggtttacaatg

SEQ ID NO: 45 - DNA, 20 bp, Artificial Sequence
  Description of Artificial Sequence Forward Primer for SNP ID 23
gaaatgcctccctggaacag

SEQ ID NO: 46 - DNA, 19 bp, Artificial Sequence
  Description of Artificial Sequence Reverse Primer for SNP ID 23
ctctgccaagcccatcttg

SEQ ID NO: 47 - DNA, 20 bp, Artificial Sequence
  Description of Artificial Sequence Forward Primer for SNP ID 24
tccctgagcccaggtaagtc

SEQ ID NO: 48 - DNA, 21 bp, Artificial Sequence
  Description of Artificial Sequence Reverse Primer for SNP ID 24
tgttccctgatcctcatccag

SEQ ID NO: 49 - DNA, 24 bp, Artificial Sequence
  Description of Artificial Sequence Forward Primer for SNP ID 25
catcctcgtcactgactaatagcg

SEQ ID NO: 50 - DNA, 20 bp, Artificial Sequence
  Description of Artificial Sequence Reverse Primer for SNP ID 25
tcaacagcgaactccacctg

SEQ ID NO: 51 - DNA, 20 bp, Artificial Sequence
  Description of Artificial Sequence Forward Primer for SNP ID 26
gccagggactgaagctgaac

SEQ ID NO: 52 - DNA, 21 bp, Artificial Sequence
  Description of Artificial Sequence Reverse Primer for SNP ID 26
aaagcatcagtgggcagaatc

SEQ ID NO: 53 - DNA, 24 bp, Artificial Sequence
  Description of Artificial Sequence Forward Primer for SNP ID 27
tggtgagtggtgaggtattagcag

SEQ ID NO: 54 - DNA, 22 bp, Artificial Sequence
  Description of Artificial Sequence Reverse Primer for SNP ID 27
cactacatggcacctcaggaag

SEQ ID NO: 55 - DNA, 21 bp, Artificial Sequence
  Description of Artificial Sequence Forward Primer for SNP ID 28
agggattcagtcagttccgag

SEQ ID NO: 56 - DNA, 24 bp, Artificial Sequence
  Description of Artificial Sequence Reverse Primer for SNP ID 28
cgttacttccaaatgtcaggagtg

SEQ ID NO: 57 - DNA, 24 bp, Artificial Sequence
  Description of Artificial Sequence Forward Primer for SNP ID 29
cactttgagcactctcaggagaac

SEQ ID NO: 58 - DNA, 20 bp, Artificial Sequence
  Description of Artificial Sequence Reverse Primer for SNP ID 29
gctttgagcaaggcttccag

SEQ ID NO: 59 - DNA, 20 bp, Artificial Sequence
  Description of Artificial Sequence Forward Primer for SNP ID 30
gagaacgggctgaggacaag

SEQ ID NO: 60 - DNA, 20 bp, Artificial Sequence
  Description of Artificial Sequence Reverse Primer for SNP ID 30
tgccagagaaagggtgactg

SEQ ID NO: 61 - DNA, 24 bp, Artificial Sequence
  Description of Artificial Sequence Forward Primer for SNP ID 31
ccctgagtctagctcaaatctctc

SEQ ID NO: 62 - DNA, 21 bp, Artificial Sequence
  Description of Artificial Sequence Reverse Primer for SNP ID 31
cctgctccttgagcttgtcac

SEQ ID NO: 63 - DNA, 20 bp, Artificial Sequence
  Description of Artificial Sequence Forward Primer for SNP ID 32
ctgagggtcccttcaccaag

SEQ ID NO: 64 - DNA, 22 bp, Artificial Sequence
  Description of Artificial Sequence Reverse Primer for SNP ID 32
gcaacagcctgaatgtacacag

SEQ ID NO: 65 - DNA, 20 bp, Artificial Sequence
  Description of Artificial Sequence Forward Primer for SNP ID 33
ctgagggtcccttcaccaag

SEQ ID NO: 66 - DNA, 22 bp, Artificial Sequence
  Description of Artificial Sequence Reverse Primer for SNP ID 33
gcaacagcctgaatgtacacag

SEQ ID NO: 67 - DNA, 20 bp, Artificial Sequence
  Description of Artificial Sequence Forward Primer for SNP ID 34
ctgagggtcccttcaccaag

SEQ ID NO: 68 - DNA, 22 bp, Artificial Sequence
  Description of Artificial Sequence Reverse Primer for SNP ID 34
gcaacagcctgaatgtacacag

SEQ ID NO: 69 - DNA, 20 bp, Artificial Sequence
  Description of Artificial Sequence Forward Primer for SNP ID 35
ctgagggtcccttcaccaag

SEQ ID NO: 70 - DNA, 22 bp, Artificial Sequence
  Description of Artificial Sequence Reverse Primer for SNP ID 35
gcaacagcctgaatgtacacag

SEQ ID NO: 71 - DNA, 20 bp, Artificial Sequence
  Description of Artificial Sequence Forward Primer for SNP ID 36
ccactgtcctggctcagatg

SEQ ID NO: 72 - DNA, 21 bp, Artificial Sequence
  Description of Artificial Sequence Reverse Primer for SNP ID 36
gaggatgtcacggttccagtc

SEQ ID NO: 73 - DNA, 24 bp, Artificial Sequence
  Description of Artificial Sequence Forward Primer for SNP ID 37
gtgaccttcctctgtcctattacg

SEQ ID NO: 74 - DNA, 20 bp, Artificial Sequence
  Description of Artificial Sequence Reverse Primer for SNP ID 37
tttcagcagggacagagtcg

SEQ ID NO: 75 - DNA, 24 bp, Artificial Sequence
  Description of Artificial Sequence Forward Primer for SNP ID 38
gtgaccttcctctgtcctattacg

SEQ ID NO: 76 - DNA, 20 bp, Artificial Sequence
  Description of Artificial Sequence Reverse Primer for SNP ID 38
tttcagcagggacagagtcg

SEQ ID NO: 77 - DNA, 24 bp, Artificial Sequence
  Description of Artificial Sequence Forward Primer for SNP ID 39
gtgaccttcctctgtcctattacg

SEQ ID NO: 78 - DNA, 20 bp, Artificial Sequence
  Description of Artificial Sequence Reverse Primer for SNP ID 39
tttcagcagggacagagtcg

SEQ ID NO: 79 - DNA, 20 bp, Artificial Sequence
  Description of Artificial Sequence Forward Primer for SNP ID 40
atccactggccattctgctg

SEQ ID NO: 80 - DNA, 20 bp, Artificial Sequence
  Description of Artificial Sequence Reverse Primer for SNP ID 40
gctcaaggcagactggtgtc

SEQ ID NO: 81 - DNA, 20 bp, Artificial Sequence
  Description of Artificial Sequence Forward Primer for SNP ID 41
aggcagacaaatcgccactc

SEQ ID NO: 82 - DNA, 20 bp, Artificial Sequence
  Description of Artificial Sequence Reverse Primer for SNP ID 41
tgcatgggcttcagtagagc

SEQ ID NO: 83 - DNA, 20 bp, Artificial Sequence
  Description of Artificial Sequence Forward Primer for SNP ID 42
aggcagacaaatcgccactc

SEQ ID NO: 84 - DNA, 20 bp, Artificial Sequence
  Description of Artificial Sequence Reverse Primer for SNP ID 42
tgcatgggcttcagtagagc

SEQ ID NO: 85 - DNA, 20 bp, Artificial Sequence
  Description of Artificial Sequence Forward Primer for SNP ID 43
tgtgggctgctctgaggtag

SEQ ID NO: 86 - DNA, 20 bp, Artificial Sequence
  Description of Artificial Sequence Reverse Primer for SNP ID 43
cccaccctcctttggtaatg

SEQ ID NO: 87 - DNA, 20 bp, Artificial Sequence
  Description of Artificial Sequence Forward Primer for SNP ID 44
gggaagacccagccataatc

SEQ ID NO: 88 - DNA, 21 bp, Artificial Sequence
  Description of Artificial Sequence Reverse Primer for SNP ID 44
gagttggtgggcactaaggtg

SEQ ID NO: 89 - DNA, 20 bp, Artificial Sequence
  Description of Artificial Sequence Forward Primer for SNP ID 45
gggaagacccagccataatc

SEQ ID NO: 90 - DNA, 21 bp, Artificial Sequence
  Description of Artificial Sequence Reverse Primer for SNP ID 45
gagttggtgggcactaaggtg

SEQ ID NO: 91 - DNA, 20 bp, Artificial Sequence
  Description of Artificial Sequence Forward Primer for SNP ID 46
ctgggcctgtgtcttcactg

SEQ ID NO: 92 - DNA, 21 bp, Artificial Sequence
  Description of Artificial Sequence Reverse Primer for SNP ID 46
ggcaaaggtcttggtgtcaac

SEQ ID NO: 93 - DNA, 24 bp, Artificial Sequence
  Description of Artificial Sequence Forward Primer for SNP ID 47
gcagccctctgactatatgagttg

SEQ ID NO: 94 - DNA, 20 bp, Artificial Sequence
  Description of Artificial Sequence Reverse Primer for SNP ID 47
agaacgcagcaaggaagcac

SEQ ID NO: 95 - DNA, 23 bp, Artificial Sequence
  Description of Artificial Sequence Forward Primer for SNP ID 48
gattagcgtttctttcagccatc

SEQ ID NO: 96 - DNA, 22 bp, Artificial Sequence
  Description of Artificial Sequence Reverse Primer for SNP ID 48
tctgaattcccattcttcatgc

SEQ ID NO: 97 - DNA, 20 bp, Artificial Sequence
  Description of Artificial Sequence Forward Primer for SNP ID 49
ggccaaaggttccaggagag

SEQ ID NO: 98 - DNA, 21 bp, Artificial Sequence
  Description of Artificial Sequence Reverse Primer for SNP ID 49
cgatgcagagactgtccagag

SEQ ID NO: 99 - DNA, 20 bp, Artificial Sequence
  Description of Artificial Sequence Forward Primer for SNP ID 50
ggccaaaggttccaggagag

SEQ ID NO: 100 - DNA, 21 bp, Artificial Sequence
  Description of Artificial Sequence Reverse Primer for SNP ID 50
cgatgcagagactgtccagag

SEQ ID NO: 101 - DNA, 20 bp, Artificial Sequence
  Description of Artificial Sequence Forward Primer for SNP ID 51
cctcctcagtttctccagcg

SEQ ID NO: 102 - DNA, 19 bp, Artificial Sequence
  Description of Artificial Sequence Reverse Primer for SNP ID 51
tgggcatctgaatggaagc

SEQ ID NO: 103 - DNA, 20 bp, Artificial Sequence
  Description of Artificial Sequence Forward Primer for SNP ID 52
accaatccaagggctaggtg

SEQ ID NO: 104 - DNA, 22 bp, Artificial Sequence
  Description of Artificial Sequence Reverse Primer for SNP ID 52
caggtccagcagtgatccatac

SEQ ID NO: 105 - DNA, 23 bp, Artificial Sequence
  Description of Artificial Sequence Forward Primer for SNP ID 53
gttacaaacctgacttgtggctc

SEQ ID NO: 106 - DNA, 20 bp, Artificial Sequence
  Description of Artificial Sequence Reverse Primer for SNP ID 53
ggctatgagttcccgctcag

SEQ ID NO: 107 - DNA, 22 bp, Artificial Sequence
  Description of Artificial Sequence Forward Primer for SNP ID 54
gccaaacaatccctcatgatac

SEQ ID NO: 108 - DNA, 20 bp, Artificial Sequence
  Description of Artificial Sequence Reverse Primer for SNP ID 54
atgcttcctctaccatggcg

SEQ ID NO: 109 - DNA, 22 bp, Artificial Sequence
  Description of Artificial Sequence Forward Primer for SNP ID 55
tcccaactcatttcagcatctc

SEQ ID NO: 110 - DNA, 20 bp, Artificial Sequence
  Description of Artificial Sequence Reverse Primer for SNP ID 55
tgtctgcctccctgactctg

SEQ ID NO: 111 - DNA, 20 bp, Artificial Sequence
  Description of Artificial Sequence Forward Primer for SNP ID 56
ttaactggccctgtctggtg

SEQ ID NO: 112 - DNA, 20 bp, Artificial Sequence
  Description of Artificial Sequence Reverse Primer for SNP ID 56
gtgcacacagaggtgtagcg

SEQ ID NO: 113 - DNA, 20 bp, Artificial Sequence
  Description of Artificial Sequence Forward Primer for SNP ID 57
gggcttcttctgcatgtgtg

SEQ ID NO: 114 - DNA, 20 bp, Artificial Sequence
  Description of Artificial Sequence Reverse Primer for SNP ID 57
tgcttcccactgttctcagc

SEQ ID NO: 115 - DNA, 22 bp, Artificial Sequence
  Description of Artificial Sequence Forward Primer for SNP ID 58
aaacctcactgtctgcttcctg

SEQ ID NO: 116 - DNA, 20 bp, Artificial Sequence
  Description of Artificial Sequence Reverse Primer for SNP ID 58
caggtgagatcggcacactc

SEQ ID NO: 117 - DNA, 23 bp, Artificial Sequence
  Description of Artificial Sequence Forward Primer for SNP ID 59
ccacctgtaagaacagaagtggc

SEQ ID NO: 118 - DNA, 20 bp, Artificial Sequence
  Description of Artificial Sequence Reverse Primer for SNP ID 59
acccaagtttgggactctgc

SEQ ID NO: 119 - DNA, 20 bp, Artificial Sequence
  Description of Artificial Sequence Forward Primer for SNP ID 60
tttggccttgtttgcctctg

SEQ ID NO: 120 - DNA, 20 bp, Artificial Sequence
  Description of Artificial Sequence Reverse Primer for SNP ID 60
aaggccacagtttgagaacg

SEQ ID NO: 121 - DNA, 22 bp, Artificial Sequence
  Description of Artificial Sequence Forward Primer for SNP ID 61
gagtgtggtccataaacttggc

SEQ ID NO: 122 - DNA, 20 bp, Artificial Sequence
  Description of Artificial Sequence Reverse Primer for SNP ID 61
accacgtctctagccagtcg

SEQ ID NO: 123 - DNA, 20 bp, Artificial Sequence
  Description of Artificial Sequence Forward Primer for SNP ID 62
gctgtgtgacgttaggccag

SEQ ID NO: 124 - DNA, 20 bp, Artificial Sequence
  Description of Artificial Sequence Reverse Primer for SNP ID 62
agatactgggttccatccgc

SEQ ID NO: 125 - DNA, 20 bp, Artificial Sequence
  Description of Artificial Sequence Forward Primer for SNP ID 63
gttctcggaggtggctcttg

SEQ ID NO: 126 - DNA, 22 bp, Artificial Sequence
  Description of Artificial Sequence Reverse Primer for SNP ID 63
ccacatcactctctcctgcatc

SEQ ID NO: 127 - DNA, 20 bp, Artificial Sequence
  Description of Artificial Sequence Forward Primer for SNP ID 64
gcttattcctgcaaggcgtc

SEQ ID NO: 128 - DNA, 20 bp, Artificial Sequence
  Description of Artificial Sequence Reverse Primer for SNP ID 64
aatggaagccaaaggcacag

SEQ ID NO: 129 - DNA, 20 bp, Artificial Sequence
  Description of Artificial Sequence Forward Primer for SNP ID 65
gcttattcctgcaaggcgtc

SEQ ID NO: 130 - DNA, 20 bp, Artificial Sequence
  Description of Artificial Sequence Reverse Primer for SNP ID 65
aatggaagccaaaggcacag

SEQ ID NO: 131 - DNA, 20 bp, Artificial Sequence
  Description of Artificial Sequence Forward Primer for SNP ID 66
gcttattcctgcaaggcgtc

SEQ ID NO: 132 - DNA, 20 bp, Artificial Sequence
  Description of Artificial Sequence Reverse Primer for SNP ID 66
aatggaagccaaaggcacag

SEQ ID NO: 133 - DNA, 20 bp, Artificial Sequence
  Description of Artificial Sequence Forward Primer for SNP ID 67
aaccctgagcctgtcacctg

SEQ ID NO: 134 - DNA, 20 bp, Artificial Sequence
  Description of Artificial Sequence Reverse Primer for SNP ID 67
tgagccctgaatgcgagtag

SEQ ID NO: 135 - DNA, 20 bp, Artificial Sequence
  Description of Artificial Sequence Forward Primer for SNP ID 68
tgtgaccttcctggctcttc

SEQ ID NO: 136 - DNA, 20 bp, Artificial Sequence
  Description of Artificial Sequence Reverse Primer for SNP ID 68
agcctcactgacatgccttg

SEQ ID NO: 137 - DNA, 21 bp, Artificial Sequence
  Description of Artificial Sequence Forward Primer for SNP ID 69
caactgtgagtgaccgtggag

SEQ ID NO: 138 - DNA, 23 bp, Artificial Sequence
  Description of Artificial Sequence Reverse Primer for SNP ID 69
agtgaggtattggaatctgaggc

SEQ ID NO: 139 - DNA, 21 bp, Artificial Sequence
  Description of Artificial Sequence Forward Primer for SNP ID 70
caatattagctccaccgaggc

SEQ ID NO: 140 - DNA, 21 bp, Artificial Sequence
  Description of Artificial Sequence Reverse Primer for SNP ID 70
cctcgccaactaaatgcagac

SEQ ID NO: 141 - DNA, 22 bp, Artificial Sequence
  Description of Artificial Sequence Forward Primer for SNP ID 71
cataagccgagtggtacagagc

SEQ ID NO: 142 - DNA, 23 bp, Artificial Sequence
  Description of Artificial Sequence Reverse Primer for SNP ID 71
tccaaaggccatagtttaccaag

SEQ ID NO: 143 - DNA, 20 bp, Artificial Sequence
  Description of Artificial Sequence Forward Primer for SNP ID 72
tggcttgaggttctggcttc

SEQ ID NO: 144 - DNA, 20 bp, Artificial Sequence
  Description of Artificial Sequence Reverse Primer for SNP ID 72
tgtgacgggtaaggcagatg

SEQ ID NO: 145 - DNA, 21 bp, Artificial Sequence
  Description of Artificial Sequence Forward Primer for SNP ID 73
cctatgctcagccaaggtcag

SEQ ID NO: 146 - DNA, 20 bp, Artificial Sequence
  Description of Artificial Sequence Reverse Primer for SNP ID 73
agaaccacctgggctgctac

SEQ ID NO: 147 - DNA, 21 bp, Artificial Sequence
  Description of Artificial Sequence Forward Primer for SNP ID 74
cctatgctcagccaaggtcag

SEQ ID NO: 148 - DNA, 20 bp, Artificial Sequence
  Description of Artificial Sequence Reverse Primer for SNP ID 74
agaaccacctgggctgctac

SEQ ID NO: 149 - DNA, 20 bp, Artificial Sequence
  Description of Artificial Sequence Forward Primer for SNP ID 75
ctgtgatgggctgcagaatg

SEQ ID NO: 150 - DNA, 20 bp, Artificial Sequence
  Description of Artificial Sequence Reverse Primer for SNP ID 75
ggagagcctccagttcaagc

SEQ ID NO: 151 - DNA, 21 bp, Artificial Sequence
  Description of Artificial Sequence Forward Primer for SNP ID 76
cacccagtgcagccttatagc

SEQ ID NO: 152 - DNA, 20 bp, Artificial Sequence
  Description of Artificial Sequence Reverse Primer for SNP ID 76
acctccctctctgccttctg

SEQ ID NO: 153 - DNA, 20 bp, Artificial Sequence
  Description of Artificial Sequence Forward Primer for SNP ID 77
accacggagtctggcatcac

SEQ ID NO: 154 - DNA, 24 bp, Artificial Sequence
  Description of Artificial Sequence Reverse Primer for SNP ID 77
cggtcagaacaaagagagtggaac

SEQ ID NO: 155 - DNA, 20 bp, Artificial Sequence
  Description of Artificial Sequence Forward Primer for SNP ID 78
tttgtccttgggcttggtag

SEQ ID NO: 156 - DNA, 22 bp, Artificial Sequence
  Description of Artificial Sequence Reverse Primer for SNP ID 78
cagggagaggtatacgatggtg

SEQ ID NO: 157 - DNA, 22 bp, Artificial Sequence
  Description of Artificial Sequence Forward Primer for SNP ID 79
cccatcccgttaaagcacttag

SEQ ID NO: 158 - DNA, 20 bp, Artificial Sequence
  Description of Artificial Sequence Reverse Primer for SNP ID 79
aggatgggcttcccactcag

SEQ ID NO: 159 - DNA, 22 bp, Artificial Sequence
  Description of Artificial Sequence Forward Primer for SNP ID 80
cccatcccgttaaagcacttag

SEQ ID NO: 160 - DNA, 20 bp, Artificial Sequence
  Description of Artificial Sequence Reverse Primer for SNP ID 80
aggatgggcttcccactcag

SEQ ID NO: 161 - DNA, 24 bp, Artificial Sequence
  Description of Artificial Sequence Forward Primer for SNP ID 81
gtgtgctttgtttggtttgcatag

SEQ ID NO: 162 - DNA, 19 bp, Artificial Sequence
  Description of Artificial Sequence Reverse Primer for SNP ID 81
ctgggaatgtgccagcaag

SEQ ID NO: 163 - DNA, 24 bp, Artificial Sequence
  Description of Artificial Sequence Forward Primer for SNP ID 82
gtgtgctttgtttggtttgcatag

SEQ ID NO: 164 - DNA, 19 bp, Artificial Sequence
  Description of Artificial Sequence Reverse Primer for SNP ID 82
ctgggaatgtgccagcaag

SEQ ID NO: 165 - DNA, 24 bp, Artificial Sequence
  Description of Artificial Sequence Forward Primer for SNP ID 83
gtgtgctttgtttggtttgcatag

SEQ ID NO: 166 - DNA, 19 bp, Artificial Sequence
  Description of Artificial Sequence Reverse Primer for SNP ID 83
ctgggaatgtgccagcaag

SEQ ID NO: 167 - DNA, 20 bp, Artificial Sequence
  Description of Artificial Sequence Forward Primer for SNP ID 84
ccgtgggaacatcctctgtg

SEQ ID NO: 168 - DNA, 20 bp, Artificial Sequence
  Description of Artificial Sequence Reverse Primer for SNP ID 84
gggtttgcagaatcagcctc

SEQ ID NO: 169 - DNA, 21 bp, Artificial Sequence
  Description of Artificial Sequence Forward Primer for SNP ID 85
ggcacacttgagcacttgatg

SEQ ID NO: 170 - DNA, 21 bp, Artificial Sequence
  Description of Artificial Sequence Reverse Primer for SNP ID 85
ggaggacacacagaggaatgc

SEQ ID NO: 171 - DNA, 23 bp, Artificial Sequence
  Description of Artificial Sequence Forward Primer for SNP ID 86
caaacaggtcacatttgctgaag

SEQ ID NO: 172 - DNA, 22 bp, Artificial Sequence
  Description of Artificial Sequence Reverse Primer for SNP ID 86
tggcccacacagactaataagc

SEQ ID NO: 173 - DNA, 23 bp, Artificial Sequence
  Description of Artificial Sequence Forward Primer for SNP ID 87
caaacaggtcacatttgctgaag

SEQ ID NO: 174 - DNA, 22 bp, Artificial Sequence
  Description of Artificial Sequence Reverse Primer for SNP ID 87
tggcccacacagactaataagc

SEQ ID NO: 175 - DNA, 20 bp, Artificial Sequence
  Description of Artificial Sequence Forward Primer for SNP ID 88
ggaggcctgacagccatatc

SEQ ID NO: 176 - DNA, 21 bp, Artificial Sequence
  Description of Artificial Sequence Reverse Primer for SNP ID 88
gccatatgtggaacaagcagc

SEQ ID NO: 177 - DNA, 21 bp, Artificial Sequence
  Description of Artificial Sequence Forward Primer for SNP ID 89
ttctttctgccatcaagttgc

SEQ ID NO: 178 - DNA, 20 bp, Artificial Sequence
  Description of Artificial Sequence Reverse Primer for SNP ID 89
gctttgccaggagcctagtg

SEQ ID NO: 179 - DNA, 22 bp, Artificial Sequence
  Description of Artificial Sequence Forward Primer for SNP ID 90
tgtgatcttccaattcctcctg

SEQ ID NO: 180 - DNA, 20 bp, Artificial Sequence
  Description of Artificial Sequence Reverse Primer for SNP ID 90
tatggcagggaaggaagcac

SEQ ID NO: 181 - DNA, 20 bp, Artificial Sequence
  Description of Artificial Sequence Forward Primer for SNP ID 91
tggtgaagctgctggatgac

SEQ ID NO: 182 - DNA, 24 bp, Artificial Sequence
  Description of Artificial Sequence Reverse Primer for SNP ID 91
gacacccaccaaagcatgtataac

SEQ ID NO: 183 - DNA, 20 bp, Artificial Sequence
  Description of Artificial Sequence Forward Primer for SNP ID 92
tgcaccagacagggtagctg

SEQ ID NO: 184 - DNA, 346 bp, Artificial Sequence
  Description of Artificial Sequence Reverse Primer for SNP ID 92
ccatccagccaagtccttgtagtgcaccagacagggtagctgccatccagccaagtccttgtagccagacggcttagagcactgcgagagcatccaccagagtgggacggatgaagatgaacgcatcccagcagccctcttagcttgccttgaacttgctctgccacacctgccctttattggtctctccagcaggtacaggcacgccttcatctctgcaagctccctcatgtcctggtgcattgagctgcgaagagagccagaggatcacaggtcgtaggcagatgccatccagactgggtcagtgctccatgtgggaatcggtgtcaaggcacatcacatggtc

SEQ ID NO: 185 - DNA, 20 bp, Artificial Sequence
  Description of Artificial Sequence Forward Primer for SNP ID 93
tgcaccagacagggtagctg

SEQ ID NO: 186 - DNA, 22 bp, Artificial Sequence
  Description of Artificial Sequence Reverse Primer for SNP ID 93
ccatccagccaagtccttgtag

SEQ ID NO: 187 - DNA, 20 bp, Artificial Sequence
  Description of Artificial Sequence Forward Primer for SNP ID 94
ccagacggcttagagcactg

SEQ ID NO: 188 - DNA, 20 bp, Artificial Sequence
  Description of Artificial Sequence Reverse Primer for SNP ID 94
cgagagcatccaccagagtg

SEQ ID NO: 189 - DNA, 20 bp, Artificial Sequence
  Description of Artificial Sequence Forward Primer for SNP ID 95
ggacggatgaagatgaacgc

SEQ ID NO: 190 - DNA, 20 bp, Artificial Sequence
  Description of Artificial Sequence Reverse Primer for SNP ID 95
atcccagcagccctcttagc

SEQ ID NO: 191 - DNA, 20 bp, Artificial Sequence
  Description of Artificial Sequence Forward Primer for SNP ID 96
ttgccttgaacttgctctgc

SEQ ID NO: 192 - DNA, 21 bp, Artificial Sequence
  Description of Artificial Sequence Reverse Primer for SNP ID 96
cacacctgccctttattggtc

SEQ ID NO: 193 - DNA, 20 bp, Artificial Sequence
  Description of Artificial Sequence Forward Primer for SNP ID 97
tctccagcaggtacaggcac

SEQ ID NO: 194 - DNA, 20 bp, Artificial Sequence
  Description of Artificial Sequence Reverse Primer for SNP ID 97
gccttcatctctgcaagctc

SEQ ID NO: 195 - DNA, 20 bp, Artificial Sequence
  Description of Artificial Sequence Forward Primer for SNP ID 98
cctcatgtcctggtgcattg

SEQ ID NO: 196 - DNA, 20 bp, Artificial Sequence
  Description of Artificial Sequence Reverse Primer for SNP ID 98
agctgcgaagagagccagag

SEQ ID NO: 197 - DNA, 22 bp, Artificial Sequence
  Description of Artificial Sequence Forward Primer for SNP ID 99
gatcacaggtcgtaggcagatg

SEQ ID NO: 198 - DNA, 20 bp, Artificial Sequence
  Description of Artificial Sequence Reverse Primer for SNP ID 99
ccatccagactgggtcagtg

SEQ ID NO: 199 - DNA, 19 bp, Artificial Sequence
  Description of Artificial Sequence Forward Primer for SNP ID 100
ctccatgtgggaatcggtg

SEQ ID NO: 200 - DNA, 20 bp, Artificial Sequence
  Description of Artificial Sequence Reverse Primer for SNP ID 100
tcaaggcacatcacatggtc